(19) United States
(12) Patent Application Publication
(10) Pub. No.: US 2019/0107391 A1
(43) Pub. Date: Apr. 11, 2019

(54) TOOLS, SYSTEMS AND METHODS FOR STANDARDIZATION OF BRA CUP MEASUREMENTS

(71) Applicants: Bradley BENGTSON, Grand Rapids, MI (US); Caroline GLICKSMAN, Brielle, NJ (US)

(72) Inventors: Bradley BENGTSON, Grand Rapids, MI (US); Caroline GLICKSMAN, Brielle, NJ (US)

(21) Appl. No.: 16/195,491

(22) Filed: Nov. 19, 2018

Related U.S. Application Data
(63) Continuation of application No. 13/939,688, filed on Jul. 11, 2013, now abandoned.
(60) Provisional application No. 61/670,312, filed on Jul. 11, 2012.

(51) Int. Cl. G01B 21/02; A41C 5/00; A61B 5/107; A61B 5/00; A41H 1/00

(52) U.S. Cl. CPC G01B 21/02; A41C 5/00; A61B 5/1079; A61B 5/1072; A41C 3/0092; A61B 5/4312; A41H 1/00

(57) ABSTRACT

Disclosed is an algorithmic measurement data tool, method and system of tools used to standardize bra cup sizing, that can be useful to women, surgeons, and bra manufacturers to provide consistency and realistic expectations of breast and standardize bra cup sizes. The method utilizes measurement of the breast hemicircumference, used in conjunction with a standardized scale developed according to measurement results obtained from over 5000 patients, to accurately determine the woman's cup size. Tools, such as a slide rule, a software program, and 3D imaging may also be used, establishing the breast hemicircumference, to determine the proper bra size. The tool documents and algorithmically compares documented hemicircumference measurements to actual reported cup sizes and manufacturers. This tool, method and system is particularly useful for breast surgery patients, providing a standardized bra cup measurement of the patient's current size and a more accurate estimation of what her post-operative result may be.

### RELATED APPLICATIONS

This application is a continuation of application Ser. No. 13/939,688 filed 11 Jul. 2013 which claims the benefit of U.S. provisional patent application Ser. No. 61/670,312 filed 11 Jul. 2012.

### BACKGROUND OF THE INVENTION

Most potential breast surgery patients and every plastic surgeon understand there is no standard bra cup size. However, preoperative and postoperative bra cup size remains the primary terminology and determination of expected breast size by most patients. This creates a significant conundrum for the patient and the plastic surgeon. During a breast augmentation or revision consultation, after discussing safety and cost, the discussion comes down to outcome. The patient questions what she will look like, and seeks to find a balance between being too big and too small and satisfaction with her size following surgery. Breast patients have specific expectations with regards to bra cup size, and failing to meet real or unrealistic expectations with regard to cup size postoperatively is the leading cause of patient dissatisfaction and even occasional litigation. In addition breast implant size change remains a primary cause for reoperation and revisional breast surgery, particularly in breast augmentation patients.

Patients are often very specific as to what bra cup size they wish their postoperative size to be, for example, “I would like to be a full C.” Patients are often disappointed when, post-operatively, they find that they fit into a different cup size than anticipated. Multiple problems exist without a standard sizing system. A patient's determination of what size they want to be is based upon a manufacturer or brand of bra they typically wear, which can vary in size and fit without any standard. In addition, they have never been the specific size they are following surgery and have no reference point. A 36C bra from Victoria's Secret is quite different than that exact size in a Bali, Maidenform and Warner's bra. Most surgeons and patients understand that different manufacturers create different size bras, however manufacturers also design bras with demi and full cup coverage, varying fabrics, padding, elastics, as well as the “sister size issue”, such as a 34 C and 36 B having a similar fit. Multiple methods to bypass this cup size challenge and provide an accurate and realistic prediction and sense of post-operative size have been attempted, but all attempts fall short. In addition in order to give a patient an idea of her postoperative size, even without a known bra cup size, Surgeons have tried stuffing bras with implants, but devices may appear a minimum of one implant size smaller when placed inside the body, so placing implants inside a bra or on the chest with a tight spandex shirt may give a general visual idea but is not very accurate. The patient's individual breast tissues and shape also contribute significantly to patient outcomes. Searching for a prior augmentation patient, with photographs, with a similar breast shape and volume who had an augmentation comparable to what the patient in front of the surgeon desires is challenging, time consuming, frustrating, and again does not answer the question of determining a standard post-operative bra cup size.

Therefore, there remains an unmet need for a standardized tool, system and method to standardize bra cup sizing which can be utilized by patients, surgeons and bra manufacturers, and especially to provide an accurate bra cup size and representation for breast surgery patients' post-operative size. It also will be possible to compare bra cup sizes between manufacturers.

### SUMMARY OF THE INVENTION

The present invention provides a simplified and standardized bra cup sizing system based on over 5000 patient evaluations and measurements, compared to documentation of patient reported bra cup size by manufacturer, with the goal of standardizing bra cup measurements. Data collection and this measurement tool and algorithm, initiated and devised over 15 years ago, is based on the breast hemicircumference measurement only for the specific determination of a patient's bra cup size which is set and based upon actual data for accuracy. This tool, method and system are independent of the under bust and full breast, overbreast measurements which are the present, primary measurements for sizing, as shown in FIG. 1. Although the band size must be known for a proper bra fit, it is not used in the determination of cup size as with other systems. In the commonly used method, cup size is determined by subtracting the band size from the over-bust measurement. In the most common sizing system, the difference between the two equals the cup size, for example 0 inches=AA, 1 inch=A, 2 inches=B, 3 inches C, and so on. The new system will help standardize bra cup sizing between the leading bra manufacturers in the United States and help create a more uniform sizing language between surgeons and their patients, providing patients with a more accurate awareness of their current size in order to more accurately estimate and select their desired size, therefore increasing patient satisfaction and decreasing size change operations and litigation.

This new tool, method and system based on specific measured data and reported breast size has been placed into an algorithm with specific measurements obtained from a patients direct breast measurements obtained in multiple ways either as a direct measurement with a tape measure, malleable ruler, or new 3-D imaging and simulation systems such as Canfield VECTRA. These measurements are placed into a measurement tool, such as a slide-rule format, computer software, or smartphone program, and used to generate a specific bra cup size by manufacturer when entered.

In addition, the present invention could be integrated into a 3D imaging system or handheld measurement tool and used to perform standardized bra measurements commercially such as at a Victoria Secret or bra/lingerie store or manufacturer and the data set and measurement tool may also be used for patient/client specific bra or garment manufacturing specific to each patient.

Further these measurements could be sent to bra or garment manufacturers and individual unique garments prepared for each client/patient.

### BRIEF DESCRIPTION OF THE DRAWINGS

FIG. 1 is a view of the prior art method of measuring the bust to determine bra band and cup size.

FIG. 2 is a front view showing the measurement method according to the present invention.

FIG. 3 is an alternate view of the measurement method shown in FIG. 2.

FIG. 4 is one embodiment of a slide rule tool used to determine the cup size, based on the hemicircumference measurement determined by the method shown in FIGS. 2 and 3.

FIG. 5 is an alternative view of the embodiment of a slide rule tool shown in FIG. 4.

FIG. 6A is an alternative view of the embodiment of a slide rule tool shown in FIG. 4.

FIG. 6B is an alternative view of the embodiment of a slide rule tool shown in FIG. 4.

FIG. 7 shows one embodiment of the implementation of a software program according to the present invention on a smart phone.

FIG. 8 shows a second embodiment of the implementation of a software program according to the present invention on a computer.

FIG. 9 shows a method of measuring the breast hemicircumference according to the present invention using a 3D imaging device.

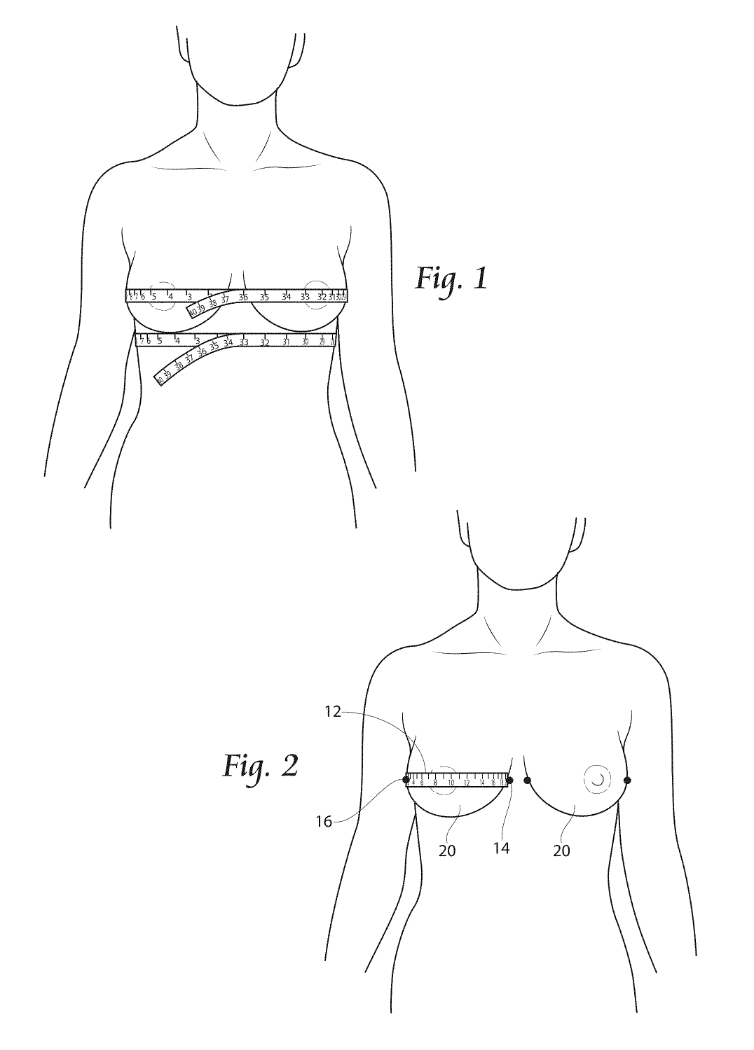

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

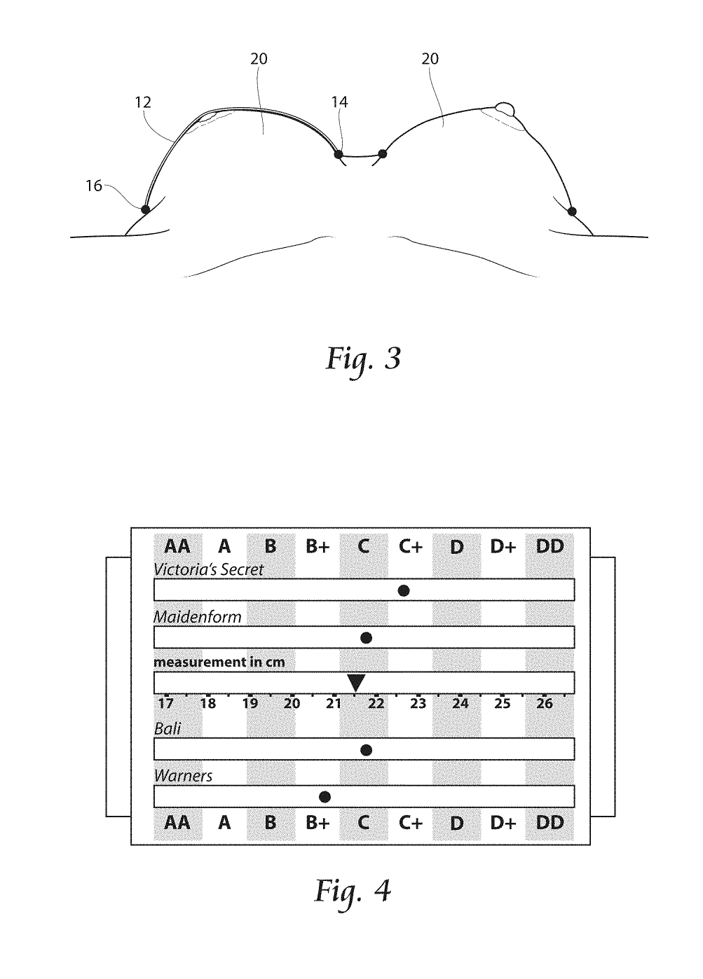

The present invention provides a novel measurement tool, method and a system for performing a breast measurement and using the measurements based on a databased algorithm, to determine a specific bra cup size, that can provide standardized sizing for manufacturers and individuals. The standardization method utilizes measurement of the breast hemicircumference (HC), using a simple soft tape measure 12, transparent ruler, or similar measurement tool, measured from the medial most point 14 of the breast 20 at nipple areolar level to the lateral most point 16 of the breast 20 over the maximum apex 18 of the breast 20. Examples of the measurement of the breast HC are shown in FIGS. 2 and 3.

Three dimensional imaging, such as Vectra 3D, may also be used to measure the HC of the breast. New 3-D Vectra imaging has helped simplify and automatically landmark and measure validated hemi-circumference distances in patients.

The breast HC measurement was used in a study of over 3000 primary augmentation patients over a 15 year period (Allergan Medical Corporation, Style 410 Cohesive Gel Implant Study). Pre-operative and post-operative breast HC was measured and compared to patent reported bra cup sizes. In addition, 450 primary augmentation patients from one surgeon's (Bengtson) cohort as a subset of this overall data was collected in a similar fashion and compared to the overall database. The average hemicircumference organized by patient reported bra cup sizes showed mean measurements listed below in Table 1:

TABLE 1

| Cup Size | Allergan Overall Data | Bengtson Cohort |
|---|---|---|
| A Cup | 17.0 cm | 16.5 cm |
| B Cup | 19.9 cm | 19.0 cm |
| C Cup | 21.4 cm | 21.3 cm |
| D Cup | 23.5 cm | 23.5 cm |
| DD Cup | 25.4 cm | 25.3 cm |

In reporting their bra cup sizes, patients also provided the bra manufacturer name. Of the data in Table 1, approximately 85% of the sizes reported were for Victoria's Secret bras, followed by Maidenform, Bali, and Calvin Klein. As such, the average measurements listed are somewhat skewed towards the correlating size in Victoria's Secret merchandise. However, the other manufacturers varied minimally from the mean HC measurement of Victoria's Secret: Of the 104 patients wearing Bali, it took more volume and a greater HC to fill a reported cup on average: 4 mm more for size B, 3 mm size C, 15 mm for size D. Calvin Klein on the other hand was significantly smaller HC averaging with 115 patients, 15 mm smaller for size A, 9 mm smaller for size B, 12 mm smaller for size C, and 12 mm smaller for D. Maidenform, with 204 patients revealed a difference of 9 mm larger for size B but all other sizes essentially the same average.

The above mean measurements collected and analyzed provide a generalized, quick check scale that may be used without referring to a reference table for a more general bra cup estimate and can provide an instant estimate based on the breast HC measurement. A surgeon, individual, or manufacturer may memorize and know instantly that approximately 17 cm is an “A cup”, 19 cm is a “B cup”, 21 cm a “C cup”, 23.5 cm a “D cup” and 25 cm a “DD cup”. For a more specific bra cup size or manufacturer size the tool or software program may be utilized.

In addition, this measurement tool and system has been validated with over 3000 additional breast measurements as related to specific manufacturer.

The present invention may also provide tools to help a woman, patient, manufacturer, surgeon, etc to use the HC measurement to accurately determine the woman's cup size, and more specifically, to determine the woman's cup size in a specific bra style by a specific manufacturer. Using the HC measurement results discussed above, a novel, data driven, algorithmic slide rule as shown in FIG. 4 can be constructed so that a person can dial in the HC measurement, as well as the bra manufacturer/style, and will be shown the appropriate cup size. The Figure is representative of the cup size differences that exists among the manufacturers, and depicts that there are differences in cup size from one manufacturer to the next. The slide rule, is thus helpful to prevent mistaken purchases based on lack of awareness of the differences between the manufacturers, and also allows patients to get started in the right size range when trying bras on that vary between both manufacturer and styles within individual brands. More importantly, it helps to standardize actual bra measurements and outcomes so patients, bra manufacturers and surgeons can be on the same page and comparing “apples to apples and oranges to oranges”. The slide rule shown in FIG. 4 is one embodiment of a slide rule according to the present invention. There are many other ways of implementing the breast HC measurements into a slide rule device, that would be known to one skilled in the art.

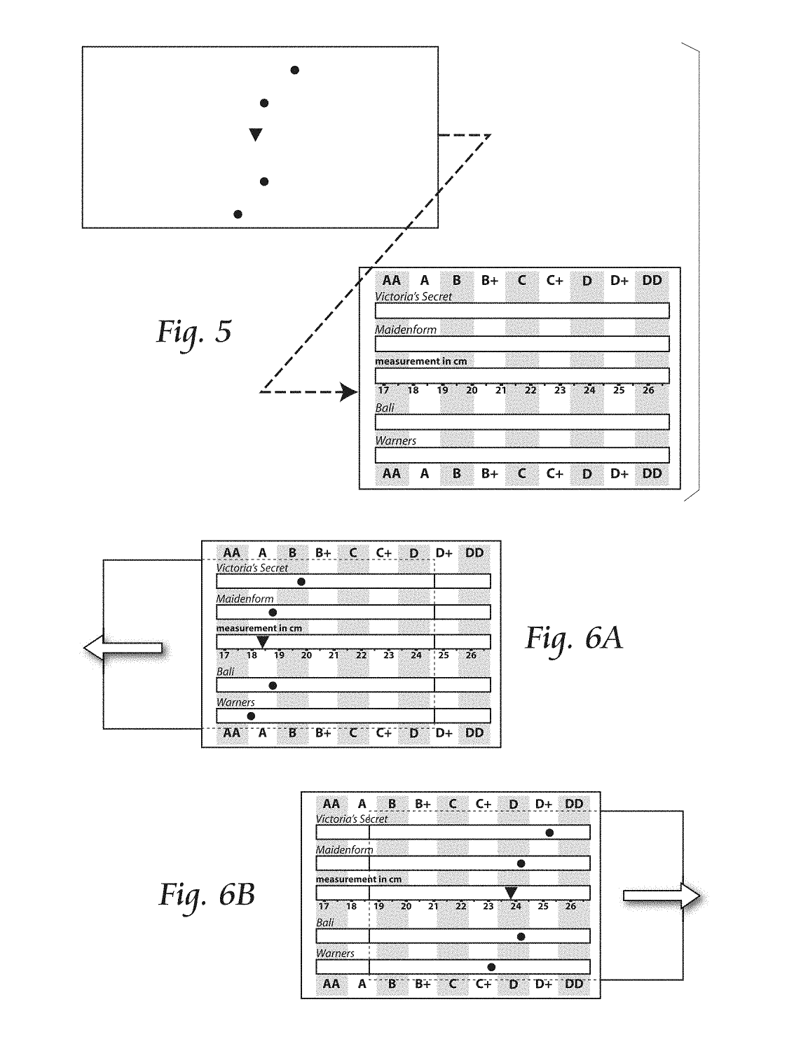

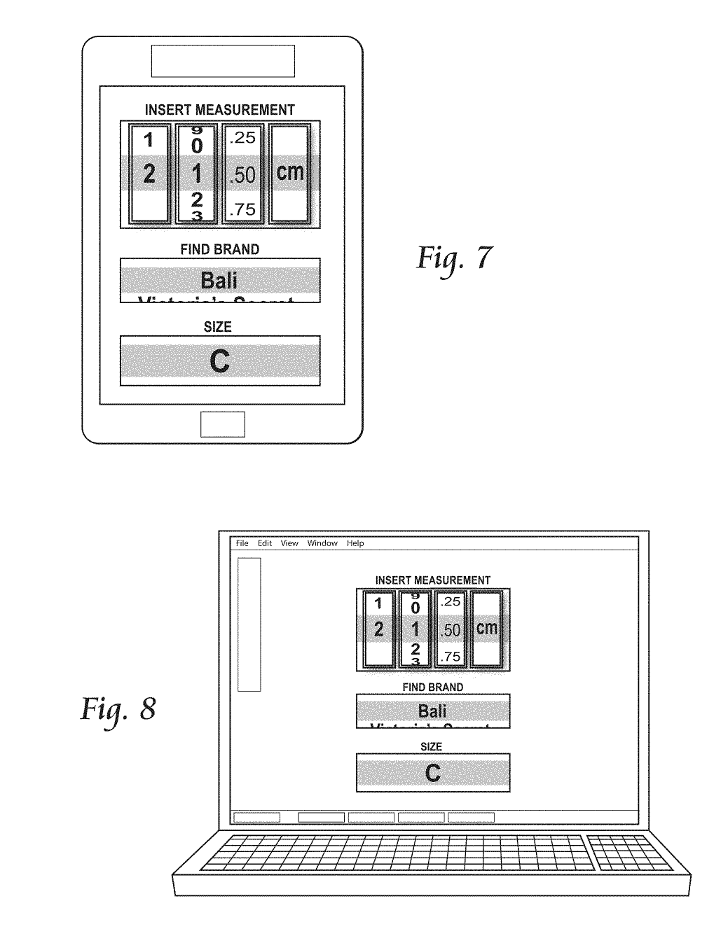

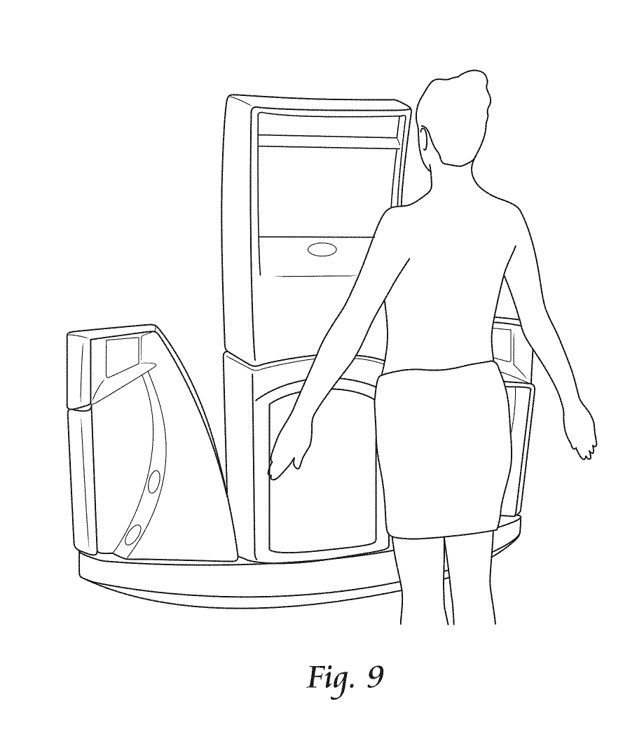

Similarly, a software program may be developed to perform the same algorithmic analysis based on each individual woman's HC measurements as discussed above. The software program may be used for personal computers or on websites, or may be developed into a smart phone or tablet application. Such an application may be used, as one example, by a woman who knows her HC measurement while shopping, so that she can input her measurement as well as the bra style that she is looking at, in order to accurately select the appropriate bra size for purchase. Example screen shots of a possible smart phone application are shown in FIGS. 5A and 5B. As with the slide rule depicted in FIG. 4, there are other embodiments of the application and other ways of presenting the information on a smart phone or tablet screen that are within the scope of the present invention.

As noted above, 3D imaging may be used to measure the breast HC and the same analysis can be performed by the imaging device to determine and be incorporated into the 3D imaging software and thus present the cup size of the imaged patient. 3D imaging can also be used in conjunction with the present measurement system to provide visualization of what the breasts will look like post-operatively at the desired size, so that the patient is well aware of the expected results and can, therefore, make more informed decisions prior to the surgery, and be on the same page size wise with her plastic surgeon.

Once the hemicircumference measurement is obtained by the 3D imaging system the data can be integrated into the simulation system and various implant sizes and volumes can be placed and the 3D system can give a bra cup size based on our data tool and information. This can be based on any or all of the breast implant manufacturers such as Allergan, Mentor or Sientra and future manufacturers.

In addition, the method can be used to determine how the difference between pre-operation and post-operation HC measurement compares to the size of the implant used. This may provide guidance for the surgeon and patient in selecting the correct implant volume to be used for the desired cup size change. For example, knowing the volume of the implant used on a patient who went from a 17 cm HC to a 21 cm HC can be useful to a surgeon who has a patient with similar expectations. The average volume difference to get a patient up or down one cup size is approximately 220 ccs.

As a result of the techniques described herein, there has been no size change operations performed in the past 3 years over 650 primary augmentations when this measurement system is combined with 3-D imaging, and patients are involved and sign-off on their desired size range that they are looking to achieve when presented in the preoperative consultation process. In addition, litigation has been averted by re-referencing patients to this standard when a patient believes or has been told they were a smaller or larger size than what our standard size measures. Thus, this new simple and standard sizing system will be of great value and a significant contribution to surgeons and patients on many levels.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

We claim:

1. A method for determining a specific bra size for a breast comprising the steps of:
measuring the breast hemicircumference from the medial most point of the breast at the nipple areolar level to the lateral most point of the breast over the maximum apex of the breast; and
comparing the hemicircumference measurement to algorithmic tabulated breast measurements.

2. The method according to claim 1, wherein the measurement is performed with the use of a soft tape measure.

3. The method according to claim 1, wherein the measurement is performed with 3D imaging software.

4. The method of claim 1 wherein the hemicircumference measurement is compared to tabulated measurements of various bra manufacturers.

5. The method of claim 4, wherein the tabulated measurements are stored on a slide rule.

6. A system for determining a specific bra size comprising:
a table containing algorithmic tabulated breast measurements.

7. The systems of claim 6, wherein the table is stored on a computer accessible medium.

8. The system of claim 6, wherein the table is incorporated into a printed medium.

9. The system of claim 8, wherein the printed medium is in the form of a slide rule device.

10. The system of claim 9, wherein the slide rule device further incorporates tabulated measurements of various bra manufacturers.

11. The system of claim 6 further comprising a measuring device.
